(19) United States
(12) Patent Application Publication
(10) Pub. No.: US 2011/0201618 A1
(43) Pub. Date: Aug. 18, 2011

(54) KMUPS INHIBITING PROLIFERATION AND OBLITERATION OF PULMONARY ARTERY

(75) Inventor: Ing-Jun CHEN, Kaohsiung City (TW)

(73) Assignee: KAOHSIUNG MEDICAL UNIVERSITY, Kaohsiung City (TW)

(21) Appl. No.: 13/095,393

(22) Filed: Apr. 27, 2011

Related U.S. Application Data
(63) Continuation-in-part of application No. 12/572,519, filed on Oct. 2, 2009.
Application No. 12/572,519 is a continuation-in-part of application No. 11/857,483, filed on Sep. 19, 2007, now abandoned.

(30) Foreign Application Priority Data
Jun. 15, 2007 (TW) 096121950

(51) Int. Cl. A61K 31/496; A61P 11/00

(52) U.S. Cl. 514/252.16

(57) ABSTRACT

An obstructive pulmonary disease inhibiting pharmaceutical composition is provided. The obstructive pulmonary disease inhibiting pharmaceutical composition includes:

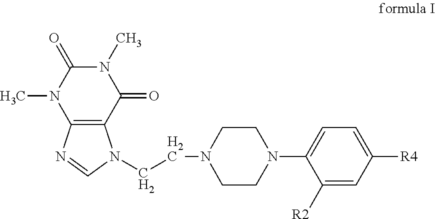

an effective amount of a compound of formula I, wherein R₂ and R₄ are each selected independently from the group consisting of a C1˜C5 alkoxy group, a hydrogen, a nitro group, and a halogen atom; and a pharmaceutically acceptable carrier.

This application is a continuation-in-part of the application Ser. No. 12/572,519, filed on Oct. 2, 2009, which is a continuation-in-part of co-pending application Ser. No. 11/857,483 filed on Sep. 19, 2007, and now abandoned, for which priority is claimed under 35 U.S.C.sctn.120; and this application claims priority of the Application No. 96121950 filed in Taiwan on Jun. 15, 2007 under 35 U.S.C.sctn.119; the entire contents of all are hereby incorporated by reference.

### FIELD OF THE INVENTION

The present invention relates to a theophylline-based moiety compound capable of enhancing the production of cGMP (cyclic guanosine monophosphate), and more particularly to those KMUPs compound capable of inhibiting MCT-induced pulmonary artery proliferation by binding to 5-HT receptors.

### BACKGROUND OF THE INVENTION

The pathological hallmark of pulmonary artery hypertension is proliferation and obliteration of small pulmonary artery (PA). KMUP-1 inhibits proliferation of pulmonary artery smooth muscle cells (PASMCs) by targeting serotonin (5-HT) receptors, enhancing endothelial NO synthase (eNOS) and transporter-mediated signaling to inhibit monocrotaline (MCT)-induced pulmonary artery hypertension (PAH). The pathogenesis of PAH is complex and multifactorial, including 5-HT-induced 5-HT transporter (5-HTT) expression and Ras homolog gene A (RhoA) activation. Recent studies of PAH-associated pulmonary artery proliferation have emphasized the role of 5-HT in pulmonary vascular remodeling, and the 5-HT/5-HTT/RhoA pathway is implicated in the mechanism of action of KMUP-1 in MCT-induced PAH, along with the NO/cGMP/RhoA pathway.

5-HT, internalized in smooth muscle through the 5-HTT, is covalently linked to RhoA by intracellular type 2 transglutaminase, leading to constitutive RhoA activation in pulmonary artery of PAH. RhoA/ROCK controls a wide variety of signal transduction pathways. In the vascular system, RhoA mediates vascular constriction by inhibiting myosin phosphatase and phosphorylation of myosin light chains.

KMUP-1 is known to inhibit PAH by cGMP-dependent inhibition of RhoA/ROCK, and could thus influence RhoA/ROCK through 5-HTT. Both the HMG-CoA reductase inhibitor simvastatin and the Rho kinase (ROCK) inhibitor Y27632 have been reported to inhibit 5-HTT and ROCK signaling in PAH-associated vascular proliferation.

5-HT acts as a mitogen in PASMCs and is important in pulmonary artery remodeling. The mitogenic effect of 5-HT is initiated by binding to one or more 5-HT receptors, including 5-HT_{2A}, 5-HT_{2B}, 5-HT_{2C} and through active 5-HTT internalized into the cell. We therefore tested whether KMUP-1 displaces radioligand binding to 5-HT_{2A}, 5-HT_{2B} and 5-HT_{2C} subreceptors. It is well known that 5-HT invokes pulmonary artery remodeling in the form of neointimal thickening of pulmonary artery, leading to pulmonary artery obstruction and PAH. Plasma levels of 5-HT are elevated in hypoxia-induced PAH. A correlation between high plasma 5-HT levels and pulmonary resistance has been reported. MCT-induced 5-HT release in plasma in this study was critical for pulmonary artery cell proliferation and PAH.

The Extracellular Signal-Regulated Protein Kinase (ERK) 1/2 and AKT kinase pathways are required for the 5-HT-induced mitogenesis of cultured PASMC. AKT (serine/threonine protein kinase) signaling plays important roles in vascular smooth muscles (VSMCs) mediating cell survival, proliferation and the migration of VSMCs induced by 5-HT. Increases of [Ca²⁺]i cause PASMCs contraction by activating myosin light chain kinase and also promote PASMC proliferation by activating cytoplasmic signal transduction proteins that are directly or indirectly involved in promoting cell proliferation. [Ca²⁺] in PASMCs is regulated by 5-HT, the key cellular element in the pathogenesis of pulmonary artery proliferation. An increase of [Ca²⁺]i caused by 5-HT promotes PASMCs proliferation by inducing immediate early genes, including cfos and c-jun, and activating cytoplasmic signal transduction proteins such as Ca²⁺/Camp (cyclic adenosine monophosphate) response element binding protein (CREB) and mitogen-activated protein kinase (MAPK).

5-HT-induced relaxation is due to the release of endothelial NO followed by an increase in cyclic GMP of smooth muscle cells. 5-HT_{2B} receptor mediates pulmonary artery relaxation via NO production to inhibit 5-HT-induced pulmonary artery constriction. Endothelial 5-HT receptor was verified to mediate relaxation of pulmonary artery by using 5-HT_{2B} receptor agonists. We therefore sought for a rational combination of 5HT_{2B} receptor agonist with 5HT_{2A} and 5HT_{2C} receptor antagonist to optimize pulmonary artery relaxation.

### SUMMARY OF THE INVENTION

In accordance with an aspect of the present invention, an obstructive pulmonary disease inhibiting pharmaceutical composition is provided. The obstructive pulmonary disease inhibiting pharmaceutical composition includes:

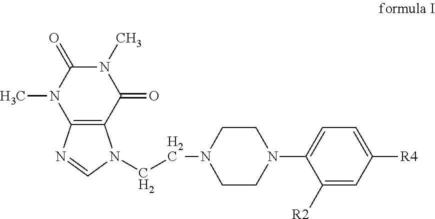

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

In accordance with another aspect of the present invention, an anti-pulmonary artery hypertension pharmaceutical composition is provided. The anti-pulmonary artery hypertension pharmaceutical composition includes:

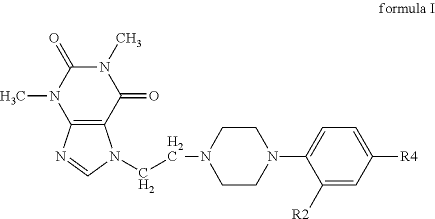

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

In accordance with a further aspect of the present invention, an vascular remodeling inhibiting pharmaceutical composition is provided. The vascular remodeling inhibiting pharmaceutical composition includes:

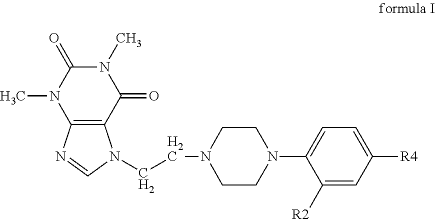

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

In accordance with further another aspect of the present invention, an obstructive pulmonary disease inhibiting pharmaceutical composition is provided. The obstructive pulmonary disease inhibiting pharmaceutical composition includes:

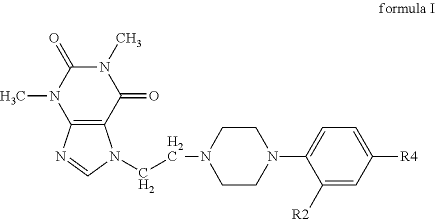

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows that KMUP-1 decreases plasma 5-HT levels in MCT-PAH;

FIG. 2 shows that KMUP-1 and simvastatin relax 5-HT-induced contraction of pulmonary artery rings;

FIG. 3 shows that Δ[Ca²⁺]i indicates the difference in [Ca²⁺]i between basal and peak levels induced by 5-HT;

FIG. 4 shows the Western blotting revealing a significant difference in expression of 5-HTT after monocrotaline (MCT) between control and KMUP-1 treatment;

FIG. 5 shows KMUP-1 inhibited 5-HT-induced 5HTT expression in PASMCs;

FIG. 6 shows Y27632 (10 μM) and KMUP-1 (1-100 μM) concentration-dependently inhibited 5-HT-induced expression of 5-HTT;

FIG. 7 shows the comparison of effects of KMUP-1 and simvastatin at 10 μM on 5-HT-induced 5-HTT expression;

FIG. 8 shows the time courses of 5-HT induced RhoA translocation and ROCK expression in PASMCs;

FIG. 9 shows the time courses of 5-HT induced ROCK expression in PASMCs;

FIG. 10 shows that KMUP-1 suppresses 5-HT-induced RhoA translocation;

FIG. 11 shows KMUP-1 and Y27632 (10 μM) inhibited 5-HT-induced ROCK expression;

FIG. 12 shows that the time courses of 5-HT-stimulated ERK by 5-HT (10 μM) were changed for the indicated time periods in PASMCs;

FIG. 13 shows that the time courses of AKT phosphorylation by 5-HT (10 μM) were changed for the indicated time periods in PASMCs;

FIG. 14 shows KMUP-1 inhibited 5-HT-induced phosphorylation of ERK1/2;

FIG. 15 shows KMUP-1 inhibited 5-HT-induced phosphorylation of AKT;

FIG. 16 shows KMUP-1 inhibited 5-HT-induced migration of PASMCs;

FIG. 17 shows that cell proliferation was determined by MTT assay;

FIG. 18 shows the effects of KMUP-1 on 5-HT_{2B} and eNOS protein expression in HPAEC;

FIG. 19 shows that KMUP-1 increases the expression of eNOS in 5-HT-treated HPAEC; and

FIG. 20 shows that KMUP-1 increases the production of NO in 5-HT-treated HPAEC.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

Preferably, a theophylline-based moiety compound derivative, i.e. KMUPs, which is obtained by reacting theophylline compound with piperazine compound and then recrystallizing the intermediate therefrom, is provided in the present invention.

Preferably, the pharmaceutical acceptable salts of formula I may be selected from one of an organic acid and an inorganic acid.

A pharmaceutical composition is provided in the present, in which the active agent is a theophylline-based moiety compound for treating a pulmonary disease, and KMUPs compound has the activities for inhibiting monocrotaline (MCT)-induced pulmonary artery proliferation by binding to 5-HT_{2A}, 5-HT_{2B} and 5-HT_{2C} receptors, increasing endothelial eNOS/5-HT_{2B} receptor expression and NO release and inhibiting 5-HTT/RhoA/ROCK expression and AKT/ERK phosphorylation. KMUPs is suggested to be useful in the treatment of 5-HT-induced pulmonary artery proliferation.

A theophylline-based moiety compound derivative, i.e. KMUPs, which is obtained by reacting theophylline compound with piperazine compound and then recrystallizing the intermediate therefrom, is provided in the present invention. KMUPs compound has the activities for treating a pulmonary disease and the benefits of good solubility, low toxicity and safety.

Preferably, The pharmaceutical composition includes one of a KMUPs compound having a formula I or its salts

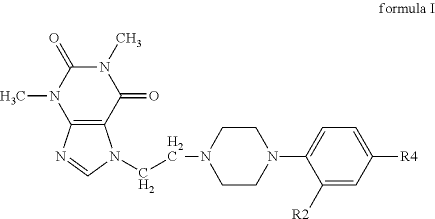

wherein R₂ and R₄ are each selected independently from the group consisting of a C1˜C5 alkoxy group, a hydrogen, a nitro group, and a halogen atom. The above-mentioned halogen refers to fluorine, chlorine, bromine and iodine.

Preferably, in one embodiment, the compound of formula I is KMUP-1, wherein R₂ is chloro atom and R₄ is hydrogen, which has the generally chemical name 7-[2-[4-(2-chlorophenyl)piperazinyl]ethyl]-1,3-dimethylxanthine. The compound of formula I is KMUP-2, wherein R₂ is methoxy group and R₄ is hydrogen, which has the chemical name 7-[2-[4-(2-methoxybenzene)piperazinyl]ethyl]-1,3-dimethylxanthine. In another embodiment, the compound of formula I also is KMUP-3, wherein R₂ is hydrogen and R₄ is nitro group, which has the chemical name 7-[2-[4-(4-nitrobenzene)piperazinyl]ethyl]-1,3-dimethylxanthine. In another embodiment, the compound of formula I also is KMUP-4, wherein R₂ is nitro group and R₄ is hydrogen, which has the chemical name 7-[2-[4-(2-nitrobenzene)piperazinyl]ethyl]-1,3-dimethylxanthine.

The compounds of formula I and the pharmaceutical acceptable salts thereof are adaptable in the clinical usage and in the human. Preferably, the pharmaceutical acceptable salts of formula I may be selected from one of an organic acid and an inorganic acid.

Preferably, the organic acid is one selected from a group consisting of a citric acid, a maleinic acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a nicotinic acid, a pentanoic acid and an aspartic acid. Preferably, the inorganic acid is one selected from a group consisting of a hydrochloride, a sulfuric acid, a phosphoric acid, a boric acid and a dihydrochloride.

The term “KMUPs compound” as used herein refers to one selected from a group consisting of KMUP-1, KMUP-2, KMUP-3, KMUP-4 and its pharmaceutical acceptable salts. Preferably, the pharmaceutical composition further includes at least one of a pharmaceutically acceptable carrier and an excipient.

To achieve the above purpose, formula I salts can be synthetically produced from the 2-Chloroethyltheophylline compound and piperazine substituted compound.

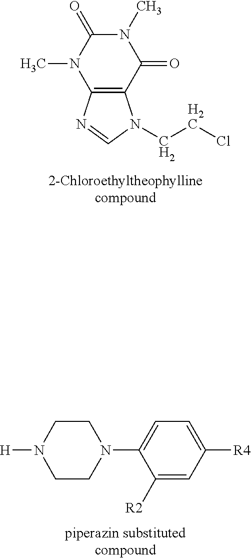

The compounds of formula I salts set forth in the examples below were prepared using the following general procedures as indicated.

The general procedure 1 includes steps of dissolving 2-Chloroethyl theophylline and piperazin substituted compound in hydrous ethanol solution, and the amount of reagent should be conjugated depending on the molecular weight percentage. After adding the strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1HCl with a white crystal.

The general procedure 2 includes steps of dissolving 2-Chloroethyl theophylline and piperazin substituted compound in hydrous ethanol solution, and the amount of reagent should be conjugated depending on the molecular weight percentage. Then, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1HCl with a white crystal.

According to the general procedure 1 or 2, KMUPs salts compound of formula I can be synthetically produced directly, from the 2-Chloroethyltheophylline compound and piperazine substituted compound. Preferably, the pharmaceutical acceptable salts of KMUP-1, KMUP-2, KMUP-3 and KMUP-4 are citric acid, nicotinic acid and hydrochloride.

In accordance with a further aspect of the present invention, depending on the desired clinical use and the effect, the adaptable administration method of KMUPs pharmaceutical composition includes one selected from a group consisting of an oral administration, an intravenous injection, a subcutaneous injection, an intraperitoneal injection, an intramuscular injection and a sublingual administration.

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-available enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier is generally used in the oral formulation. Taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

According to a further aspect of the present invention, a pharmaceutical composition is provided, in which the active agent is a theophylline-based moiety compound for inhibiting MCT-induced pulmonary artery proliferation by binding to 5-HT_{2A}, 5-HT_{2B} and 5-HT_{2C} receptors, increasing endothelial eNOS/5-HT_{2B} receptor expression and NO release and inhibiting 5-HTT/RhoA/ROCK expression and AKT/ERK phosphorylation. KMUPs is suggested to be useful in the treatment of 5-HT-induced pulmonary artery proliferation. In particular, it is related to obstructive pulmonary disease, pulmonary artery hypertension, pulmonary artery proliferation and vascular remodeling inhibiting disease.

To achieve the above purpose, the Ca²⁺ sensitivity of PASMCs is extremely important for pulmonary artery contraction. MCT-released 5-HT can increase the [Ca²⁺]i of PASMCs to enhance the vasoconstrictor response. The increase of [Ca²⁺]i promotes the proliferation of PASMC via immediate early genes. In most cases, cell proliferation requires external calcium and is inhibited when cells are cultured in a Ca²⁺-deprived or Ca²⁺ channel blocker-supplemented medium. The role of ion channels in PASMCs proliferation is currently under investigation, since the link between proliferation and ion channel activation could be a therapeutic target in clinical PAH. In PASMCs, 5-HT exerts vasoconstrictor and mitogenic effects, both dependent on an increase of cytosolic [Ca²⁺]i. 5-HT increases [Ca²⁺]i in PASMCs and causes pulmonary artery contraction, which is reduced by KMUP-1. PAH is characterized by high circulating 5-HT levels, 5-HT-induced hyper-reactivity and SMC proliferation, suggesting a major role for 5-HT in both vascular wall remodeling and elevated vascular resistance. Sustained Ca²⁺ elevation contributes to both PASMC contraction and proliferation. Increases of [Ca²⁺]i have been demonstrated to cause excessive proliferation of PASMCs in PAH, while abnormal intracellular Ca²⁺ sequestration is thought to be linked to smooth muscle dysfunction in patients with chronic obstructive pulmonary disease. We therefore suggest that the pulmonary artery relaxation and anti-proliferation effects of KMUPs include the inhibition of Ca²⁺-influx.

For proving the treating effect of pulmonary disease, the KMUPs pharmaceutically acceptable salt uses both the chronic model of PAH induced by monocrotaline (MCT) in rats, so as to confirm that KMUPs treatment would inhibit PAH, via cGMP-dependent inhibition of RhoA/ROCK II (Rho kinase II) in pulmonary artery and lung tissue.

Chronic PAH

Rats are treated with vehicle once a day for 21 days after a single intra-peritoneal injection of MCT developed PAH. Long-term daily treatment with KMUP-1 (5 mg/kg/day p.o. and 1 mg/kg/day i.p.) for 21 days significantly reduced MCT-induced increases in mean pulmonary arterial pressure (MPAP) as shown previously.

Plasma 5-HT Levels in MCT-Treated Rats

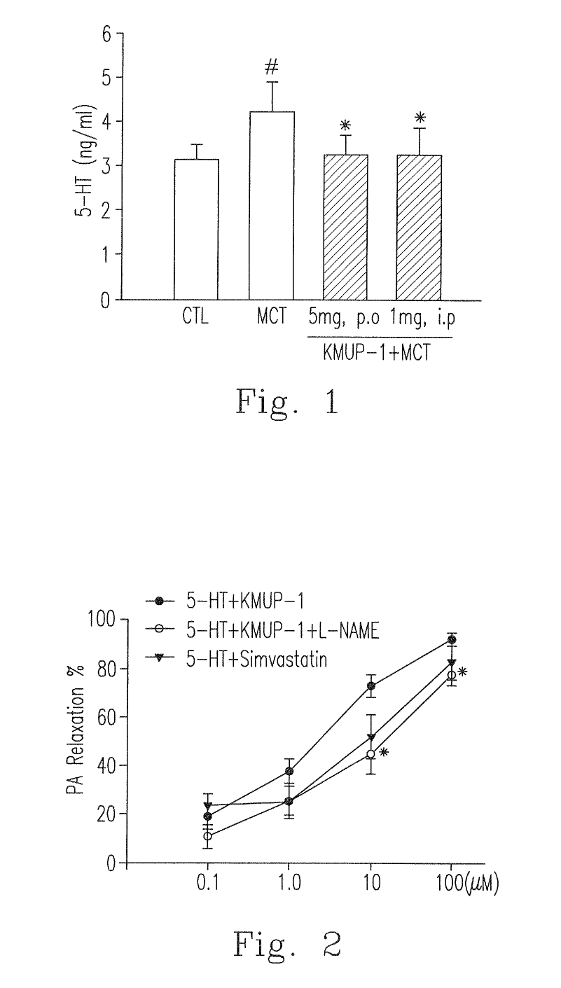

The plasma concentrations of 5-HT are shown in FIG. 1. Administration of KMUP-1 (5 mg/kg p.o., 1 mg/kg i.p.) prevents increases in plasma 5-HT levels. Data are representative of 6 experiments. The plasma concentration of 5-HT was significantly greater in MCT-PAH rats than control rats (4.2±0.7 and 3.2±0.3 ng/mL, respectively; P<0.05). The plasma concentration of 5-HT in KMUP-1-treated rats was significantly decreased, compared to non-treated MCT-PAH rats (3.2±0.5 ng/mL, p.o. and 3.2±0.6 ng/mL, i.p. 1 mg/kg, respectively; P<0.05), as shown in FIG. 1.

Effects on 5-HT-Constricted Pulmonary Artery

5-HT (10 μM) produced a contractile response in pulmonary artery of control group, which was inhibited by KMUP-1 (0.1-100 μM) and simvastatin (0.1-100 μM). In the presence of the NOS inhibitor L-NAME (100 μM), KMUP-1 (10-100 μM) reduced the maximum response of pulmonary artery to 5-HT (P<0.05). L-NAME itself did not induce any constriction, but it enhanced 5-HT-induced constriction. KMUP-1 reversed the 5-HT-induced constriction in rat pulmonary artery in a concentration-dependent manner. L-NAME did not significantly reduce this reversal, as shown in FIG. 2.

Intracellular Calcium Response to 5-HT

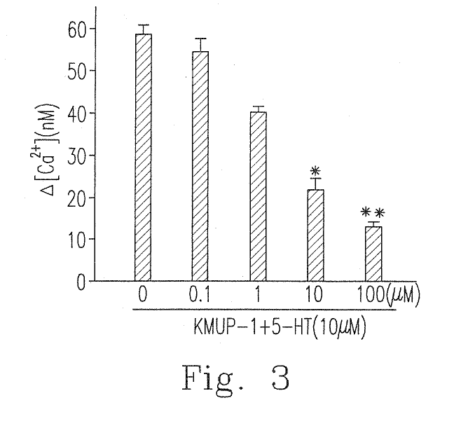

5-HT (10 μM) induced a calcium influx in PASMCs. In the following set of experiments, KMUP-1 inhibited Ca²⁺-influx induced by 5-HT in PASMCs. 5-HT (10 μM) caused a significant release of [Ca²⁺]i. KMUP-1 (0.1-100 μM) concentration-dependently inhibited elevation of [Ca²⁺]i. Δ[Ca²⁺]i indicates the difference in [Ca²⁺]i between basal and peak treated levels induced by 5-HT, as shown in FIG. 3. KMUP-1 significantly inhibited the sustained [Ca²⁺]i response to 5-HT by 65% at 10 μM.

Effect of KMUP-1 on PASMC proliferation in MCT-treated rats Medial hypertrophy was associated with an increased number of proliferating vascular cells, shown by immunohistochemistry for PCNA. In MCT-treated rats administered with vehicle, PCNA labeling indicated the proliferation of PASMCs in distal pulmonary artery walls, which were more marked in MCT-treated groups. The number of PCNA-positive cells was markedly lower in the pulmonary artery walls of rats treated with KMUP-1.

Pulmonary 5-HTT Expression and Vascular Immunochemistry

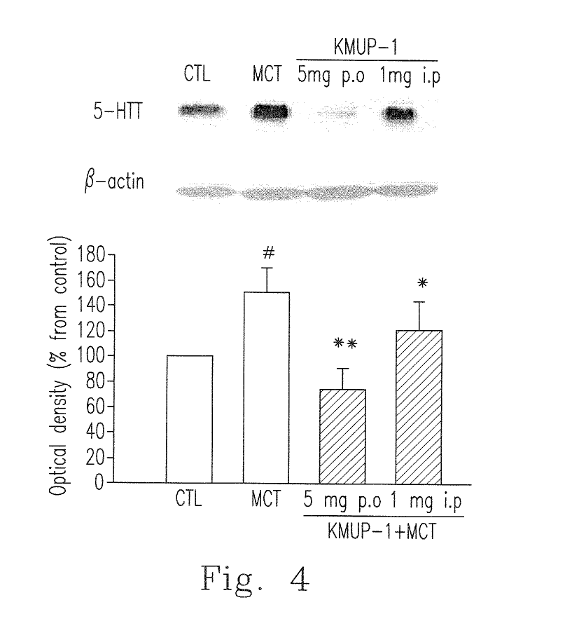

5-HTT expression in pulmonary artery 21 days after MCT injection was significantly decreased after treatment with KMUP-1 at doses of 5 mg/kg p.o. and 1 mg/kg i.p., as assayed by immunochemistry. The Western blotting measurement of 5-HTT protein expression in lung showed similar results. 5-HTT protein expression was significantly reduced by administration of KMUP-1 in lung tissue, as shown in FIG. 4.

5-HTT Expression in PASMCs

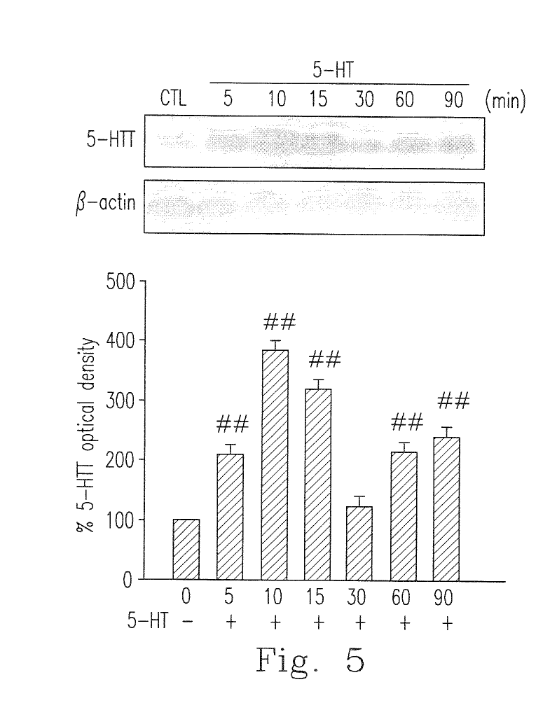

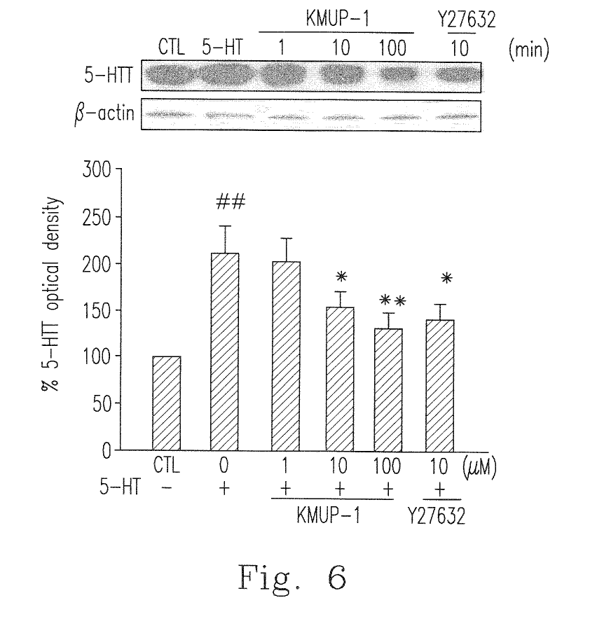

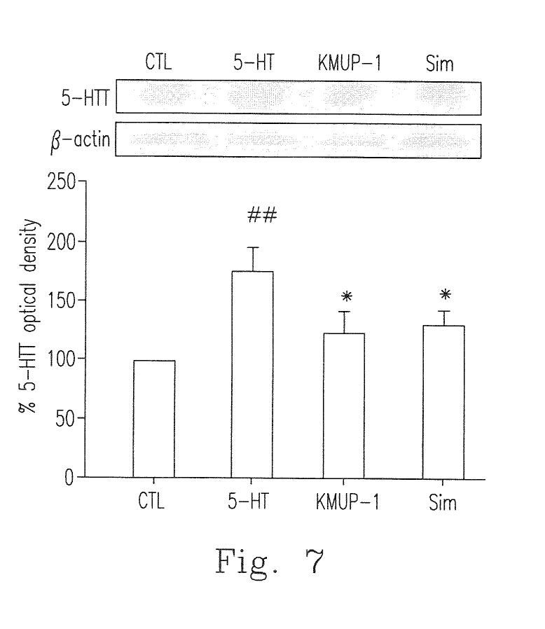

PASMCs were treated with 5-HT (10 μM) for 5-90 min. We found that 5-HTT protein expression achieved a peak 10 min after 5-HT treatment, as shown in FIG. 5. Cells were first treated with KMUP-1 (1-100 μM) and Y27632 (10 μM) for 24 h and then with 5-HT for 10 min. Y27632 (10 μM) and KMUP-1 dose-dependently inhibited 5-HT-induced expression of 5-HTT, as shown in FIG. 6. KMUP-1 and simvastatin at 10 μM also both inhibited 5-HT-induced 5-HTT expression, as shown in FIG. 7

RhoA translocation and ROCK expression in PASMCs

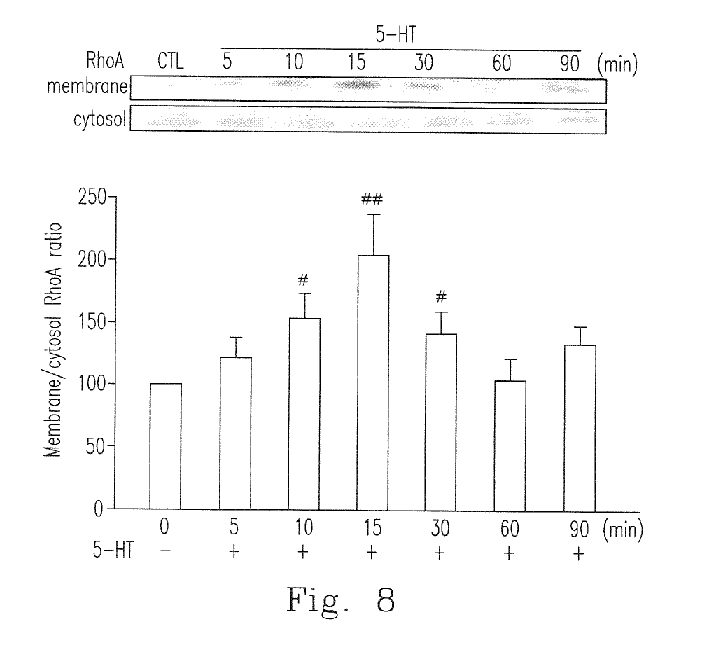

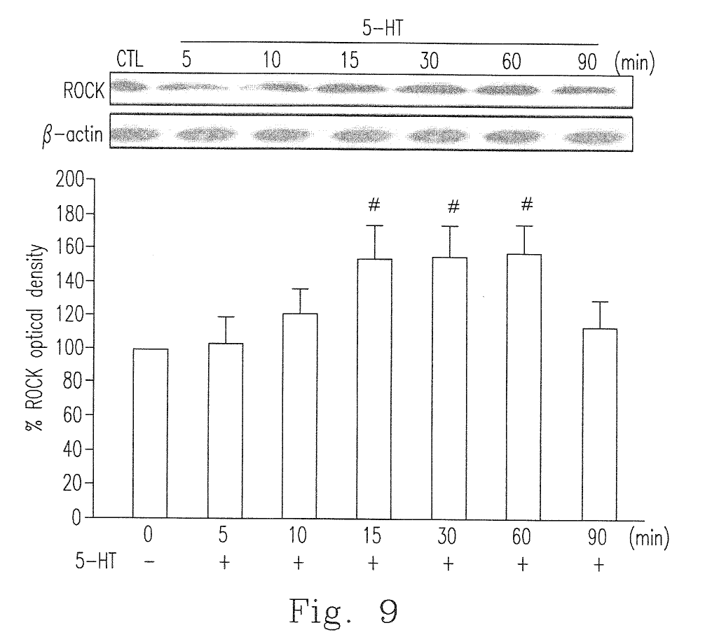

PASMCs were stimulated with 10 μM 5-HT for the indicated time periods. RhoA activity was analyzed as the membrane-to-cytosol ratio. Translocation of RhoA expression in the membrane extract was detected with anti-RhoA antibodies. 5-HT (10 μM) stimulated an increase in membrane/cytosol RhoA in PASMCs at 5 min, with a peak at 15 min and a return to basal levels by 60 min, as shown in FIG. 8. ROCK expression induced by 5-HT was significantly increased at 15-60 min and then returned to basal levels by 90 min, as shown in FIG. 9.

Attenuation of 5-HT-Induced RhoA Membrane Localization and ROCK

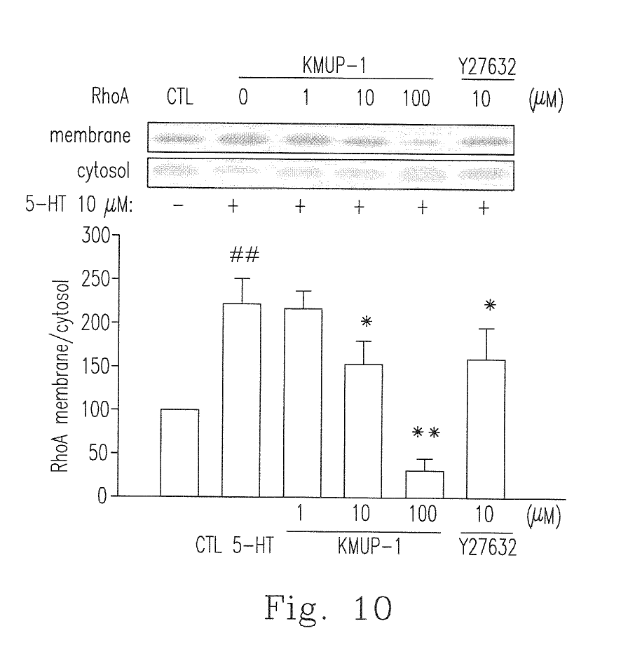

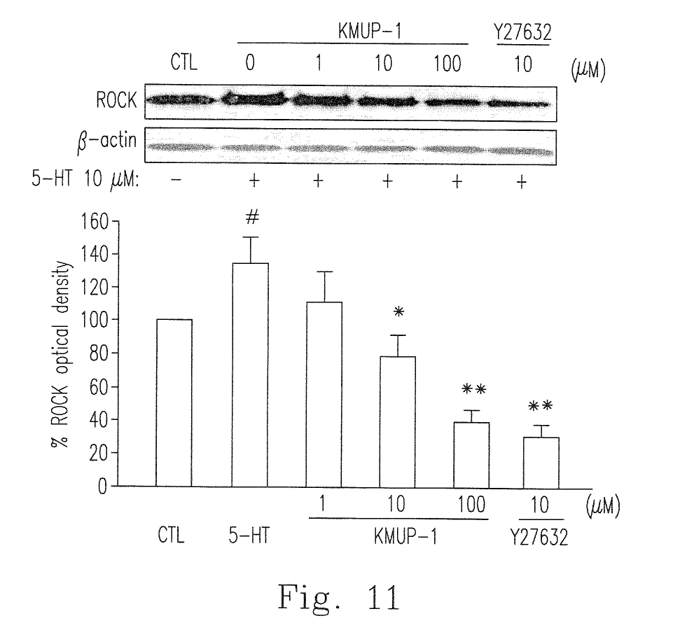

PASMCs were stimulated with 5-HT (10 μM) for 15 min prior to treatment of cells with different concentrations of KMUP-1 (1-100 μM) or Y27632 for 24 h. RhoA activation was examined by measuring the membrane-to-cytosol ratio, as shown in FIG. 10. We examined the effect of KMUP-1 on 5-HT-induced ROCK. PASMCs were stimulated with 5-HT (10 μM) for 15 min prior to treatment of cells with different concentrations of KMUP-1 or Y27632 for 24 h. Treatment with KMUP-1 (1-100 μM) significantly inhibited 5-HT-induced ROCK, as shown in FIG. 11. Activation of RhoA is associated with its translocation from the cytosol to the membrane. Stimulation of PASMCs with 5-HT (10 μM, 15 min) led to an increased level of membrane-associated RhoA. 24 h treatment with KMUP-1 dose-dependently reversed the 5-HT-induced RhoA membrane association. Y27632 (10 μM) also inhibited the membrane-tocytosol ratio of RhoA and ROCK expression.

Attenuation of 5-HT-Induced Activation of ERK1/2 and AKT

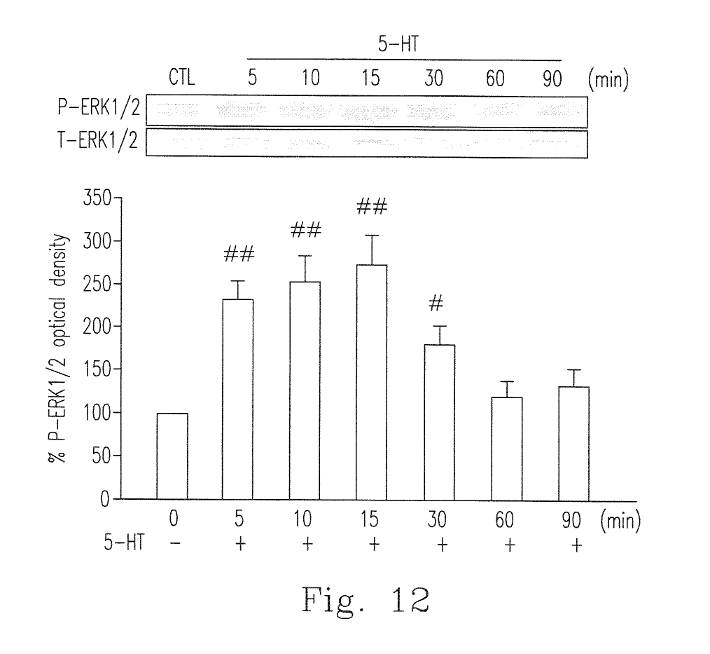

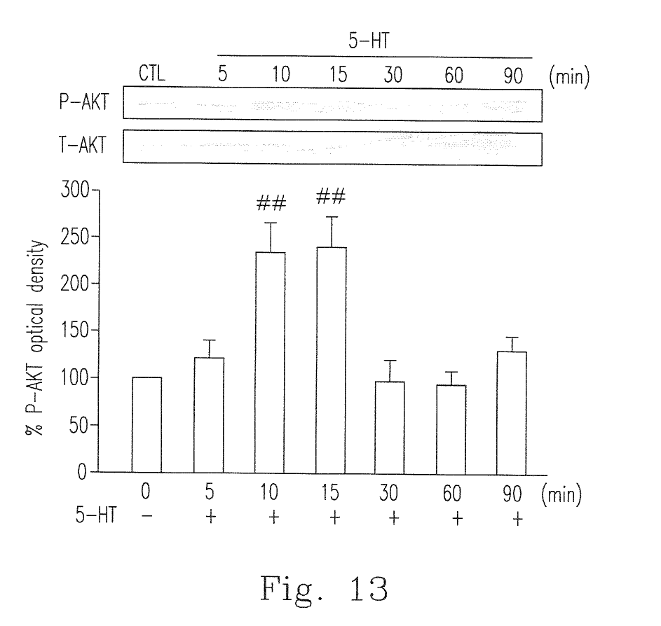

Phosphorylation or activation of ERK1/2 and AKT kinase is associated with proliferation in a variety of cell types, including PASMCs. Exposure of cells to 5-HT (10 μM) for 15 min (peak time for ERK1/2 and AKT phosphorylation) triggered 2.3-fold increases phosphorylation of ERK1/2 (FIG. 12) and AKT (FIG. 13).

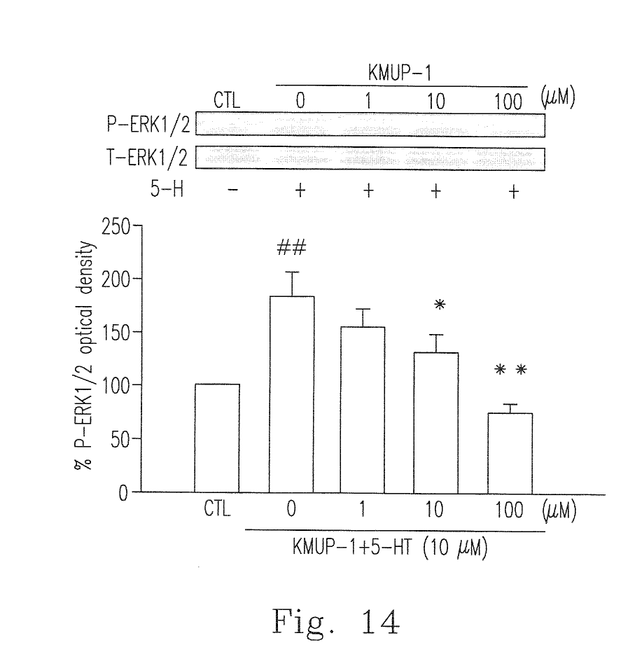

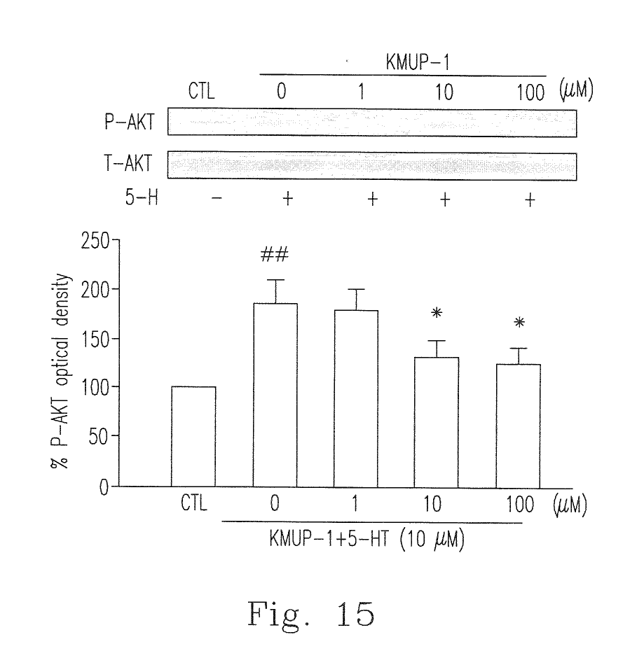

PASMCs were pre-incubated with KMUP-1 (1-100 μM) for 24 h and then stimulated with 5-HT (10 μM) for 15 min for phosphorylation of ERK1/2 and AKT. ERK1/2 and AKT phosphorylation were determined by the Western blot analysis in whole cell lysates. Sample Western blots and the bar graph indicated that KMUP-1 concentration-dependently reduced 5-HT-stimulated ERK1/2 and AKT phosphorylation (n=4-6). This response was dose-dependently inhibited by pre-incubation of cells with KMUP-1 (1-100 μM) for 24 h, as shown in FIGS. 14 and 15.

Inhibition of 5-HT-Induced PASMCs Migration and Proliferation

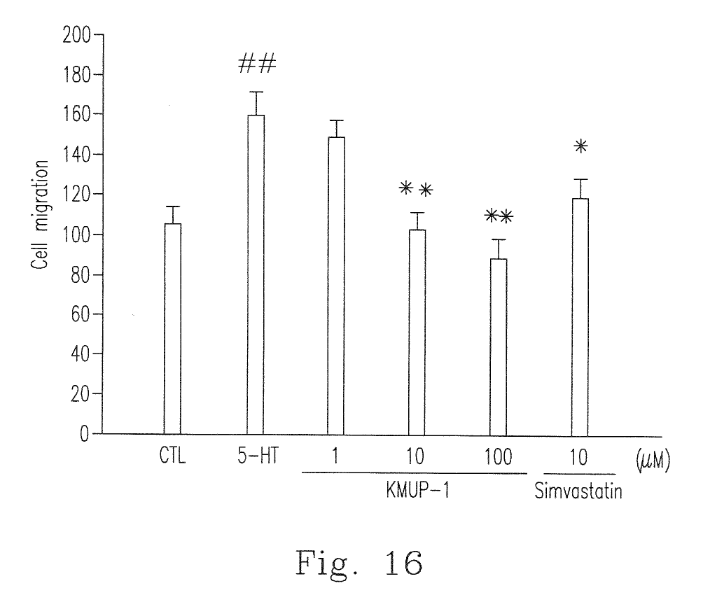

Quiescent PASMCs were pretreated with KMUP-1 (1-100 μM) and simvastatin (10 μM) for 20 min, and then incubated with 5-HT (10 μM) for 24 h. Cell migration was measured by a cell wound healing assay as described in Methods. Phase contrast images were taken 24 h after wounding. Treatment with KMUP-1 (10-100 μM) and simvastatin (10 μM) concentration-dependently suppressed 5-HT-induced PASMCs migration, as shown in FIG. 16.

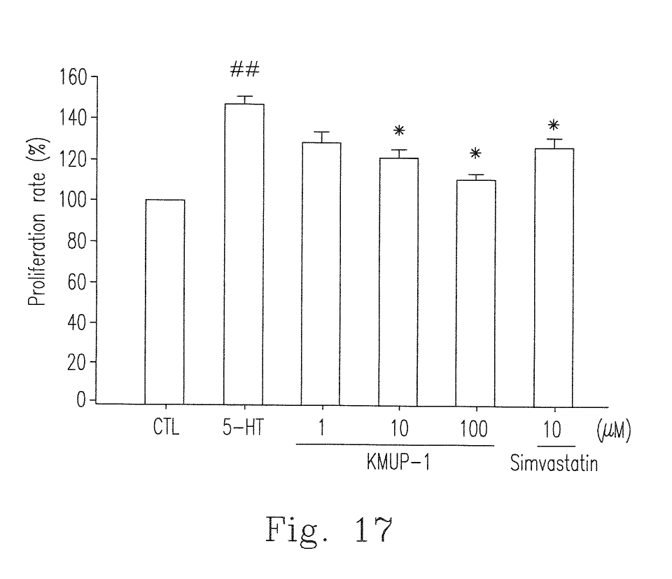

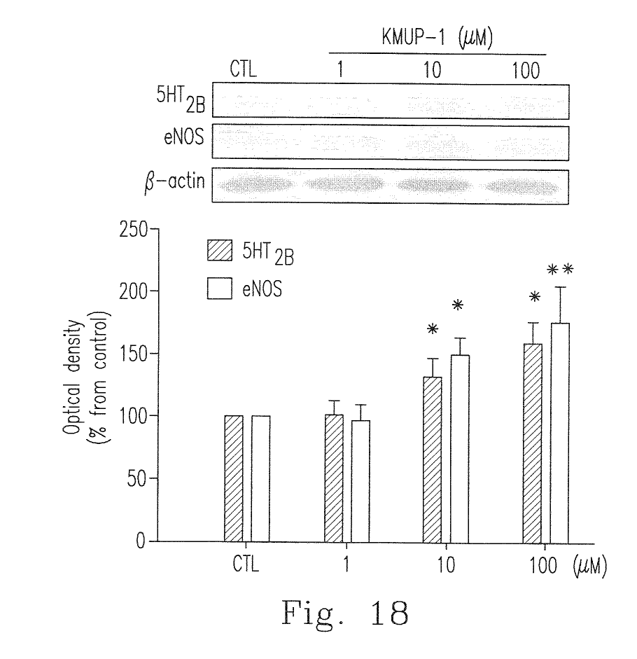

PASMCs proliferation, tested by MTT assay, was also dose-dependently inhibited by KMUP-1 and simvastatin (10 μM). Cell proliferation was determined by the MTT assay. KMUP-1 blocked 5-HT-induced PASMCs migration. Serum-starved PASMCs were stimulated with 10 μM 5-HT for 24 h with or without KMUP-1 (1-100 μM) or simvastatin (10 μM) pretreatment, as shown in FIG. 17.

Table 1 shows the estimated IC₅₀ and Kᵢ values of KMUP-1 in radioligand binding to 5-HT_{2A}, 5-HT_{2B}, and 5-HT_{2C} in human recombinant CHO-K1 cells.

TABLE 1

| | Radioligand | IC₅₀ [μM] | Kᵢ values [μM] |
|---|---|---|---|
| 5-HT_{2A} | [³H]-ketanserin | 0.34 | 0.0971 |
| 5-HT_{2B} | [³H]-LSD | 0.04 | 0.0254 |
| 5-HT_{2C} | [³H]-mesulergine | 0.408 | 0.214 |

Radioligand Binding on 5-HT_{2A}, 5-HT_{2B} and 5-HT_{2C} Receptors

As shown in Table 1, the ligand specificity of the human 5-HT_{2B} receptor to KMUP-1 was more selective than the 5-HT_{2A} and 5-HT_{2C} receptors. The affinity of these sub-receptors for KMUP-1 is 5-HT_{2A}>5-HT_{2B}>5-HT_{2C}.

The expression of eNOS, 5-HT_{2B} receptor and the production of NO in HPAEC

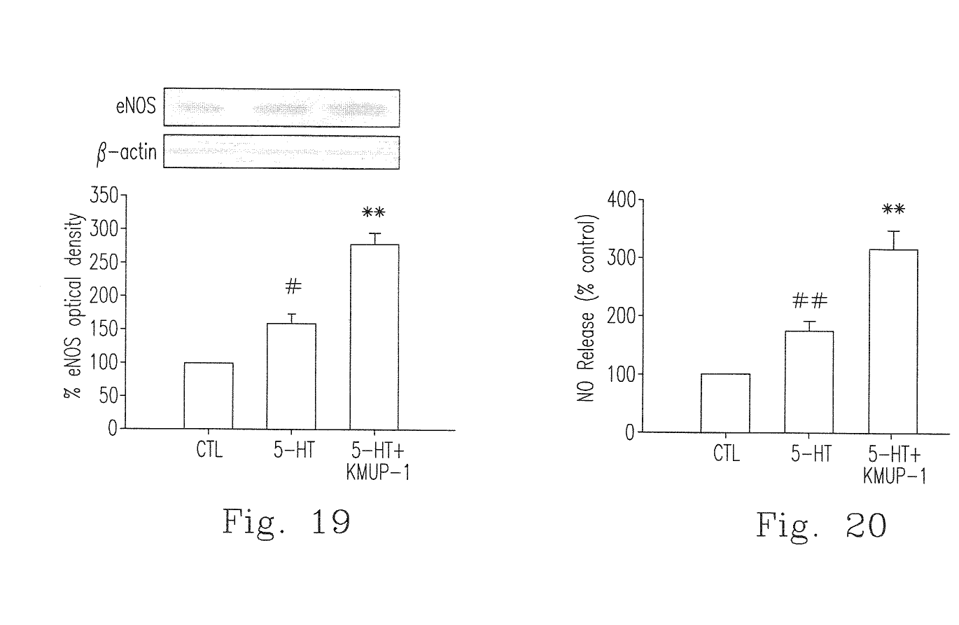

eNOS and 5-HT_{2B} receptor expressions were assessed by the Western blotting. Incubation of HPAEC with KMUP-1 (1-100 μM) for 24 h dose-dependently increased eNOS and 5-HT_{2B} receptor expressions in cultured HPAEC, as shown in FIG. 19. Incubation of 5-HT (10 μM) for 30 min in HPAEC, 5-HT increased the expression of eNOS and the release of NO, compared to the control group. Treatment of HPAEC with KMUP-1 (10 μM) for 24 h before adding 5-HT also raised the expression of eNOS and the release of NO, compared to 5-HT treatment alone, as shown in FIG. 20.

Table 2 shows the changes of protein expression of eNOS, PDE-5A and ROCKII represented by optical density (%) after application of KMUPs salts (10 μM) for 120 min, compared to the control without treatment.

TABLE 2

| Without treatment control | eNOS | PDE-5A | ROCKII |
|---|---|---|---|
| (Vehicle) | 100 (%) | 100 (%) | 100 (%) |
| KMUP-1 HCl | 155 ± 14.8 | 64 ± 4.5 | 42 ± 6.3 |
| KMUP-1-citric acid | 152 ± 13.6 | 66 ± 5.2 | 43 ± 3.8 |
| KMUP-1-nicotinic acid | 158 ± 12.4 | 62 ± 4.8 | 41 ± 2.5 |
| KMUP-2 HCl | 147 ± 8.6 | 68 ± 5.3 | 40 ± 3.7 |
| KMUP-2-citric acid | 145 ± 7.2 | 71 ± 5.4 | 40 ± 3.7 |
| KMUP-3 HCl | 148 ± 7.5 | 63 ± 5.2 | 44 ± 2.9 |
| KMUP-3-nicotinic acid | 145 ± 7.5 | 60 ± 4.1 | 43 ± 1.6 |
| KMUP-4 HCl | 135 ± 7.5 | 73 ± 3.4 | 37 ± 2.8 |
| KMUP-4-citric acid | 131 ± 6.7 | 78 ± 3.6 | 56 ± 3.4 |

P < 0.05;
significantly different from control (n = 5)

Biological Experiments

Animal Models and Hemodynamic Measurement

All experiments were performed in adult male Wistar rats (300 to 350 g) in accordance with institutional guidelines after approval by the ethical review committee. PAH development and pulmonary expression of 5-HTT were examined in rats after single injection of MCT (60 mg/kg i.p.). To assess the potential preventive effect of KMUP-1 on MCT-induced PAH and associated proliferation, we assigned rats at random to 2 groups of 8 animals which received KMUP-1 at 5 mg/kg/day p.o or 1 mg/kg/day i.p. All treatments were given once a day for 3 weeks after a single MCT injection (60 mg/kg i.p.) (Abe et al., 2004). On day 21, rats were anesthetized and pulmonary artery blood pressure (MPAP) was recorded as previously described. Lung tissues were dissected for the Western blotting and immunohistochemistry.

Reagents and Antibodies

KMUP-1 hydrochloride (KMUP-1HCl) and other KMUPs salts were synthesized in our laboratory and dissolved in distilled water. All other reagents were from Sigma (St. Louis. Mo, USA) unless otherwise specified. Anti-RhoA monoclonal antibody and anti-ERK1/2 rabbit antibody were purchased from Santa Cruz Biotechnology (CA, USA). Anti-5-HT_{2B}, anti-eNOS and anti-ROCK (ROCKII) antibody were purchased from Upstate Biotechnology (Lake Placid, N.Y., USA). Anti-5-HTT rabbit antibody was purchased from Chemicon Biotechnology (Temecula, Calif., USA). Anti-phosphor-ERK1/2, anti-AKT, anti-phospho-AKT, and horseradish peroxidase-conjugated polyclonal rabbit and mouse antibody were purchased from Santa Cruz Biotechnology. [³H] mesulergine was purchased from Amersham (Buckinghamshire, UK). [³H] ketanserin was purchased from Perkin-Elmer (Shelton, Conn., USA).

Measurement of Plasma 5-HT Levels

After MCT-treatment, rats acquired severe PAH and received sodium pentobarbital (40 mg/kg, i.p.) at day 21 for surgical anesthesia and measurement of MPAP (Chung et al., 2010). Blood samples (1.0 mL) were obtained from the heart. Blood was transferred to plastic tubes that included EDTA and was centrifuged at 100 g for 20 min. The plasma was transferred to tubes and stored at −80° C. until analysis. Plasma 5-HT levels were determined using commercially available Serotonin EIA (IBL, Minneapolis, USA). Cross-reactivity with related substances (e.g., 5-HIAA, phenylalanine, histidine and tyramine) has been reported to be less than 0.002%. Results were read from a standard curve. The threshold of detection was 0.3 ng/mL.

Isometric Force of Pulmonary Artery

Wistar rats were euthanized with an overdose of sodium pentobarbital (60 mg/kg, i.p.) before open-chest surgery. During surgery, a thoracic retractor was used to help isolate the pulmonary artery. The chest was opened to dissect the second branches of the main pulmonary artery, which were cut into 2-3 mm rings, suspended under isometric conditions and connected to a force transducer as previously described (Ugo Basile, Model 7004, Comerio-VA, Italy) to measure the constriction caused by 5-HT (10 μM). The pulmonary artery ring preparations were stretched to a basal tension of 1 g and allowed to equilibrate for 60-90 min. After equilibration, pulmonary artery rings were constricted with 5-HT (10 μM) to prime the tissues and check the functionality of the endothelium (at least 80% relaxation in response to acetylcholine 1 μM). Once the contractile response to each agonist reached a stable tension, KMUP-1 (0.1-100 μM) was cumulatively added to the organ bath in the presence of 5-HT (10 μM, pre-incubation time of 15 min). The effect of KMUP-1 on NOS and 5-HT was also studied in vessels pre-incubated with the NOS inhibitor L-NAME (100 μM) 20 min before 5-HT administration. The percentage of relaxation was estimated using the following equation: relaxation (%)=(maximal contraction−relaxation level)/(maximal contraction-basal level)×100. Data was obtained from serotonin-induced maximal contractile responses in pulmonary artery.

PASMCs Proliferation in MCT-Treated Lung Tissues

We evaluated proliferating cell nuclear antigen (PCNA) to assess PASMCs proliferation in rats treated with MCT alone or with KMUP-1. Lung tissue sections were de-paraffinized in xylene and then treated with a graded series of alcohol washes, rehydrated in PBS (pH 7.5), and incubated with target retrieval solution (DAKO Co, Tokyo, Japan) in a water bath at 90° C. for 20 min. Endogenous peroxidase activity was blocked with H₂O₂ in PBS (3%, vol/vol) for 5 min. Slides were then washed in PBS, incubated for 30 min in a protein blocking solution, and incubated for 30 min with anti-PCNA mouse monoclonal antibody (PC-10, 1:200, Dako). Antibodies were washed off, and the slides were processed with an alkaline phosphatase LSAB+system horseradish peroxidase detection kit (DAKO Co, Tokyo, Japan). Brown color was generated with a DAB substrate, and nuclei were counter-stained with hematoxylin.

Lung 5-HTT Immunohistochemical Analysis

For 5-HTT immunostaining, the lung slides were dewaxed in 100% xylene, and the sections were then rehydrated by successive immersion first in decreasing ethanol concentrations (100%, 90%, 80%, 50% and 30%) and then in water. Endogenous peroxidase activity was blocked using H₂O₂ in methanol (0.3% vol/vol) for 10 min. After three PBS washes, sections were pre-incubated in PBS supplemented with 3% (wt/vol) BSA for 30 min, and then incubated overnight at 4° C. with goat polyclonal anti-5-HTT antibody (Abcam Biotechnology, Cambridge, UK) diluted to 1:1000 in 1×PBS, 0.02% BSA. Next, the sections were exposed for 1 h to biotin-labeled anti-goat secondary antibodies (DAKO Co, Tokyo, Japan) diluted 1:1000 in the same buffer. Peroxidase staining of the slides incubated in streptavidin-biotin horseradish peroxidase solution was carried out using 3,3′-diaminobenzidine tetrahydrochloride dihydrate (DAB; DAKO Co, Tokyo, Japan) and hydrogen peroxide. Finally, the sections were stained with hematoxylin and eosin.

Preparation of PASMCS

Wistar rats were anesthetized with an overdose of sodium pentobarbital (60 mg/kg) and the skin was sterilized with 75% alcohol. The chest was opened, and the heart and lung were removed. The organs were rinsed several times in PBS. The pulmonary artery were segregated in a sterile manner. The outer sphere was peeled and the microtubule was snipped visually, and endothecia were shaved lightly 2-3 times in order to remove endothelial cells. The tunica media was prepared into scraps (1 mm³) in DMEM (Dulbecco's modified Eagle's medium). PASMCs were cultured in DMEM containing 10% fetal bovine serum (5% CO₂ at 37° C.). The culture medium was changed every 3 days and cells were subcultured until confluence. Primary cultures of 2-4 passages were used in the experiments. Cells were examined by immunofluorescence staining of α-actin to confirm the purity of PASMCs. Over 95% of the cell preparations were found to be composed of smooth muscle cells.

HPAEC Cultured

HPAEC purchased from ATCC were maintained in humidified incubator containing 5% CO₂ at 37° C. HPAEC were cultured in F-12 supplemented with 10% fetal bovine serum. The culture medium was changed every 3 days and cells were subcultured until confluence. HPAEC were harvested with a solution of trypsin-EDTA (GIBCO BRL, NY, USA) while in a logarithmic phase of growth. HPAEC were used between passages 4-8.

Microculture Tetrazolium Test (MTT)

PASMCs were seeded into 96-well plates at a density of 1×10⁴ cells/well. The cells were then incubated in medium containing vehicle (1% FBS DMEM) and 5-HT (10 μmol/L) for 24 h with or without KMUP-1 (1-100 μM) and simvastatin (10 μM) added 30 min before 5-HT. At the end of this period, MTT (2 g/L) was added to each well, and incubation is proceeded at 37° C. for 4 h. Thereafter, the medium was removed and the cells were solubilized in 150 μL DMSO. The optical density (OD) of each well was determined by enzyme-linked ELISA at 540 nm of wavelength.

PASMCs [Ca²⁺]i Measurement

The measurement of [Ca²⁺]i in PASMCs was performed using a spectrofluorophotometer as previously reported. PASMCs, cultured for 2-4 passages and re-suspended by trypsin, were loaded with Fura-2/AM for the measurement of [Ca²⁺]i changes in cells within the cuvette by spectrofluorophotometer (Shimadzu, RF-5301PC, Shimadzu, Japan). KMUP-1 was applied 5 min before application of 5-HT (10 μM).

Western Blotting

PASMCs were stimulated with 5-HT or not, as indicated. Cells were treated with KMUP-1 for 24 h before 5-HT stimulation. Whole lysates were collected and resolved by SDS-PAGE as previously described. Primary antibodies were anti-β-actin at: 1:10,000 dilution and anti-RhoA, anti-ROCK, antiphospho-ERK1/2, anti-phospho-AKT, anti-ERK1/2, anti-AKT, anti-5HTT, anti-5-HT_{2B} and anti-eNOS at 1:1000. All blots were incubated with antibodies at 4° C. overnight. After being washed, the appropriate secondary antibodies were added at a dilution of 1:1000 for 1 h at room temperature. After extensive washing, blots were developed with a Super Signal enhanced chemiluminescence kit (Biorad, Calif., USA) and visualized on Kodak AR film.

RhoA Translocation

Previous reports have shown that the active form of RhoA is translocated from the cytosol to the plasma membrane, where it activates Rho kinase. Therefore, the membrane/cytosol ratio of RhoA is considered a measure of RhoA activity. The cytosol and membrane protein of PASMCs were extracted with a CNM (cytosol, nuclear, membrane) kit. To assess membrane trans-location of RhoA, protein in membrane and cytosolic fractions was determined by the standard Western blot analysis using mouse monoclonal anti-RhoA antibody (1:1000 dilution, Santa Cruz Biotechnology) and a peroxidase-labeled anti-mouse immunoglobulin (Ig) G antibody (1:1000 dilution, Santa Cruz Biotechnology, CA, USA). The relative density of membrane to cytosolic RhoA was determined using the NIH imaging software.

Expression of 5-HTT and RhoA/ROCK and Phosphorylation of ERK1/2 and AKT Kinase

The expression of RhoA/ROCK and phosphorylation of ERK1/2 and AKT kinase in PASMCs were assessed by incubating the PASMCs with 5-HT (10 μM) for 15 min (peak time for RhoA/ROCK, ERK1/2 and AKT phosphorylation) and 5-HTT for 10 min (peak time for 5-HTT) after KMUP-1 (1-100 μM) was added to the culture of PASMCs for 24 h.

Expression of eNOS and 5-HT_{2B} Receptor in HPAEC

To measure the expression of eNOS and 5-HT_{2B} receptor in HPAEC after incubation with KMUP-1 (1-100 μM) for 24 h, whole lysates were collected for the Western blotting assay. The relative density was determined using the NIH imaging software.

Nitric Oxide Production in HPAEC

Production of NO in HPAEC was determined using the Griess method. The three step Greiss test converts nitrate (NO₃⁻) into nitrite (NO₂⁻) giving a total NO₂⁻ concentration from a standard calibration curve. HPAEC were incubated with or without KMUP-1 (10 μM) for 24 h and then incubated with 5-HT (10 μmol/L) for 30 min. Media samples from HPAEC were taken and the NO concentration was determined. Six independent experiments were carried out and data are reported as the mean mean±SEM.

Cell Migration Assay Under Microscope

Migration of PASMCs was assessed using a wound assay model in which cells grown to confluence on 6 well dishes were scraped with the edge of a fine razor. The wound edge was viewed and photographed under a microscope (Nikon, Tokyo, Japan) before and after culture for 24 h in serum-free DMEM in the presence of 5HT (10 μM). The distance that the cells migrated from the wound surface was then manually measured.

Displacement of Radioligand Binding Assay

As described previously, full length clones of 5-HT_{2A}, 5-HT_{2B} and 5-HT_{2C} receptor were prepared from CHO-K1 cells. On the basis of higher affinity with [³H]-radiolabeled ketanserin, [³H]-radiolabeled lysergic acid diethylamide (LSD) and [³H]-radiolabeled mesulergine (0.15-1.2 nM) measured, the gene products of human 5-HT_{2A} and 5-HT_{2B} receptor and 5-HT_{2C} receptor respectively were used to test KMUP-1's effects on radioligand binding ability. IC₅₀ represents the concentration of competing ligand which displaces 50% of the specific binding of the radioligand. Kᵢ values for competition curves were calculated using the Cheng-Prusoff equation (Cheng, 2001).

### Example 1

### Preparation of KMUP-2HCl salt (7-[2-[4-(2-methoxybenzene)piperazinyl]ethyl]-1,3-dimethyl xanthine HCl)

KMUP-2 (8.0 g) is dissolved in a mixture of ethanol (10 mL) and 1N HCl (60 mL) for reacting at 50° C. for 10 min. The methanol is added into the solution under room temperature and the solution is incubated over night for crystallization. The crystal is filtrated to obtain the precipitate of KMUP-2HCl salt (6.2 g).

### Example 2

### Preparation of KMUP-3HCl salt (7-[2-[4-(4-nitrobenzene)piperazinyl]ethyl]-1,3-dimethyl xanthine HCl)

KMUP-3 (8.3 g) is dissolved in a mixture of ethanol (10 mL) and 1N HCl (60 mL). The solution is reacted at 50° C. for 20 min, the methanol is added thereinto under room temperature, and the solution is incubated over night for crystallization and filtrated to obtain KMUP-3HCl salt (6.4 g).

### Example 3

### Preparation of KMUP-2-Nicotinic Acid Salt

2-Chloroethyl theophylline (8.3 g), NaOH (8.3 g) and 2-methoxy benzene-piperazin (8.3 g) are dissolved in hydrous ethanol (10 mL), and then heated under reflux for 3 h. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C., to make a saturated solution (pH 1.2). The saturated solution was sequentially treated, decolorized with activated charcoal, filtered, deposited overnight and filtered to obtain KMUP-1HCl with a white crystal.

### Example 4

### Preparation of KMUP-3-Nicotinic Acid Salt

KMUP-3 (8.3 g) is dissolved in a mixture of ethanol (10 mL) and Nicotinic acid (2.4 g). The solution is reacted at 50° C. for 20 min, the methanol is added thereinto under room temperature, and the solution is incubated over night for crystallization and filtrated to obtain KMUP-3-Nicotinic acid salt (8.3 g).

### Example 5

### Preparation of KMUP-3-Citric Acid Salt

KMUP-3 (8.3 g) is dissolved in a mixture of ethanol (10 mL) and Nicotinic acid (2.4 g). The solution is reacted at 50° C. for 20 min, the methanol is added thereinto under room temperature, and the solution is incubated over night for crystallization and filtrated to obtain KMUP-3-Citric acid salt (10.5 g).

### Example 6

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.

| | |
|---|---|
| KMUP-3 Citric acid salt | 1.05 g |
| Lactose | qs |
| Corn starch | qs |

### Embodiments

1. An obstructive pulmonary disease inhibiting pharmaceutical composition including:

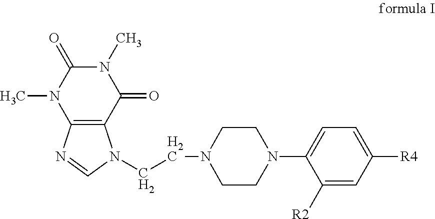

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

2. The pharmaceutical composition of Embodiment 1, wherein the halogen atom is one selected from a group consisting of a fluorine, a chlorine, a bromine and an iodine.

3. The pharmaceutical composition of any one of Embodiments 1-2, wherein the compound of formula I is KMUPs compound.

4. The pharmaceutical composition of any one of Embodiments 1-3, wherein the KMUPs compound is one selected from a group consisting of KMUP-1, KMUP-2, KMUP-3, KMUP-4 and its pharmaceutical acceptable salts.

5. An obstructive pulmonary disease inhibiting pharmaceutical composition, including:
an effective amount of a compound of 7-[2-[4-(2-chlorobenzene)piperazinyl]ethyl]-1,3-dimethylxanthine; and
a pharmaceutically acceptable carrier.

6. An obstructive pulmonary disease inhibiting pharmaceutical composition including:
an effective amount of a compound of 7-[2-[4-(2-methoxybenzene)piperazinyl]ethyl]-1,3-dimethylxanthine; and
a pharmaceutically acceptable carrier.

7. An obstructive pulmonary disease inhibiting pharmaceutical composition including:
an effective amount of a compound of 7-[2-[4-(4-nitrobenzene)piperazinyl]ethyl]-1,3-dimethylxanthine; and
a pharmaceutically acceptable carrier.

8. An obstructive pulmonary disease inhibiting pharmaceutical composition including:
an effective amount of a compound of 7-[2-[4-(2-nitrobenzene)piperazinyl]ethyl]-1,3-dimethylxanthine; and
a pharmaceutically acceptable carrier.

9. An obstructive pulmonary disease inhibiting pharmaceutical composition including:
an effective amount of a compound of 7-[2-[4-(2-chlorobenzene)piperazinyl]ethyl]-1,3-dimethylxanthine hydrochloride;
and
a pharmaceutically acceptable carrier.

10. An obstructive pulmonary disease inhibiting pharmaceutical composition including:
an effective amount of a compound of 7-[2-[4-(2-chlorobenzene)piperazinyl]ethyl]-1,3-dimethylxanthine-Citric acid; and
a pharmaceutically acceptable carrier.

11. An obstructive pulmonary disease inhibiting pharmaceutical composition including:
an effective amount of a compound of 7-[2-[4-(2-chlorobenzene)piperazinyl]ethyl]-1,3-dimethylxanthine-Nicotinic acid;
and
a pharmaceutically acceptable carrier.

12. An obstructive pulmonary disease inhibiting pharmaceutical composition including:
an effective amount of a compound of 7-[2-[4-(2-methoxybenzene)piperazinyl]ethyl]-1,3-dimethylxanthine hydrochloride; and
a pharmaceutically acceptable carrier.

13. An obstructive pulmonary disease inhibiting pharmaceutical composition including:
an effective amount of a compound of 7-[2-[4-(2-methoxybenzene)piperazinyl]ethyl]-1,3-dimethylxanthine-Citric acid;
and
a pharmaceutically acceptable carrier.

14. An obstructive pulmonary disease inhibiting pharmaceutical composition including:
an effective amount of a compound of 7-[2-[4-(2-methoxybenzene)piperazinyl]ethyl]-1,3-dimethylxanthine-Nicotinic acid; and
a pharmaceutically acceptable carrier.

15. An obstructive pulmonary disease inhibiting pharmaceutical composition including:
an effective amount of a compound of 7-[2-[4-(4-nitrobenzene)piperazinyl]ethyl]-1,3-dimethylxanthine hydrochloride;
and
a pharmaceutically acceptable carrier.

16. An obstructive pulmonary disease inhibiting pharmaceutical composition including:
an effective amount of a compound of 7-[2-[4-(4-nitrobenzene)piperazinyl]ethyl]-1,3-dimethylxanthine-Citric acid; and
a pharmaceutically acceptable carrier.

17. An obstructive pulmonary disease inhibiting pharmaceutical composition including:
an effective amount of a compound of 7-[2-[4-(4-nitrobenzene)piperazinyl]ethyl]-1,3-dimethylxanthine-Nicotinic acid;
and
a pharmaceutically acceptable carrier.

18. An obstructive pulmonary disease inhibiting pharmaceutical composition including:
an effective amount of a compound of 7-[2-[4-(2-nitrobenzene)piperazinyl]ethyl]-1,3-dimethylxanthine hydrochloride; and
a pharmaceutically acceptable carrier.

19. An obstructive pulmonary disease inhibiting pharmaceutical composition including:
an effective amount of a compound of 7-[2-[4-(2-nitrobenzene)piperazinyl]ethyl]-1,3-dimethylxanthine-Citric acid;
and
a pharmaceutically acceptable carrier.

20. An obstructive pulmonary disease inhibiting pharmaceutical composition including:
an effective amount of a compound of 7-[2-[4-(2-nitrobenzene)piperazinyl]ethyl]-1,3-dimethylxanthine-Nicotinic acid; and
a pharmaceutically acceptable carrier.

21. An anti-pulmonary artery hypertension pharmaceutical composition including:

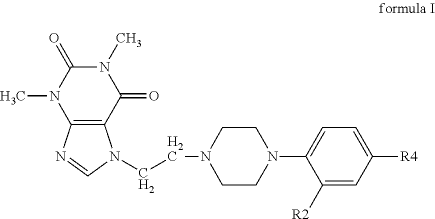

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

22. The pharmaceutical composition of Embodiment 21, wherein the halogen atom is one selected from a group consisting of a fluorine, a chlorine, a bromine and an iodine.

23. The pharmaceutical composition of any one of Embodiments 21-22, wherein the compound of formula I is KMUPs compound.

24. The pharmaceutical composition of any one of Embodiments 21-23, wherein the KMUPs compound is one selected from a group consisting of KMUP-1, KMUP-2, KMUP-3, KMUP-4 and its pharmaceutical acceptable salts.

25. The pharmaceutical composition of Embodiments 21-24, wherein the compound is for treating acute or chronic pulmonary artery hypertension.

26. An anti-pulmonary artery hypertension pharmaceutical composition, including:
an effective amount of a compound of 7-[2-[4-(2-chlorobenzene)piperazinyl]ethyl]-1,3-dimethylxanthine; and
a pharmaceutically acceptable carrier.

27. An anti-pulmonary artery hypertension pharmaceutical composition including:
an effective amount of a compound of 7-[2-[4-(2-methoxybenzene)piperazinyl]ethyl]-1,3-dimethylxanthine; and
a pharmaceutically acceptable carrier.

28. An anti-pulmonary artery hypertension pharmaceutical composition including:
an effective amount of a compound of 7-[2-[4-(4-nitrobenzene)piperazinyl]ethyl]-1,3-dimethylxanthine; and
a pharmaceutically acceptable carrier.

29. An anti-pulmonary artery hypertension pharmaceutical composition including:
an effective amount of a compound of 7-[2-[4-(2-nitrobenzene)piperazinyl]ethyl]-1,3-dimethylxanthine; and
a pharmaceutically acceptable carrier.

30. An anti-pulmonary artery hypertension pharmaceutical composition including:
an effective amount of a compound of 7-[2-[4-(2-chlorobenzene)piperazinyl]ethyl]-1,3-dimethylxanthine hydrochloride; and a pharmaceutically acceptable carrier.

31. An anti-pulmonary artery hypertension pharmaceutical composition including:
an effective amount of a compound of 7-[2-[4-(2-chlorobenzene)piperazinyl]ethyl]-1,3-dimethylxanthine-Citric acid;
and
a pharmaceutically acceptable carrier.

32. An anti-pulmonary artery hypertension pharmaceutical composition including:
an effective amount of a compound of 7-[2-[4-(2-chlorobenzene)piperazinyl]ethyl]-1,3-dimethylxanthine-Nicotinic acid;
and
a pharmaceutically acceptable carrier.

33. An anti-pulmonary artery hypertension pharmaceutical composition including:
an effective amount of a compound of 7-[2-[4-(2-methoxybenzene)piperazinyl]ethyl]-1,3-dimethylxanthine hydrochloride; and
a pharmaceutically acceptable carrier.

34. An anti-pulmonary artery hypertension pharmaceutical composition including:
an effective amount of a compound of 7-[2-[4-(2-methoxybenzene)piperazinyl]ethyl]-1,3-dimethylxanthine-Citric acid;
and
a pharmaceutically acceptable carrier.

35. An anti-pulmonary artery hypertension pharmaceutical composition including:
an effective amount of a compound of 7-[2-[4-(2-methoxybenzene)piperazinyl]ethyl]-1,3-dimethylxanthine-Nicotinic acid; and
a pharmaceutically acceptable carrier.

36. An anti-pulmonary artery hypertension pharmaceutical composition including:
an effective amount of a compound of 7-[2-[4-(4-nitrobenzene)piperazinyl]ethyl]-1,3-dimethylxanthine hydrochloride;
and
a pharmaceutically acceptable carrier.

37. An anti-pulmonary artery hypertension pharmaceutical composition including:
an effective amount of a compound of 7-[2-[4-(4-nitrobenzene)piperazinyl]ethyl]-1,3-dimethylxanthine-Citric acid; and
a pharmaceutically acceptable carrier.

38. An anti-pulmonary artery hypertension pharmaceutical composition including:
an effective amount of a compound of 7-[2-[4-(4-nitrobenzene)piperazinyl]ethyl]-1,3-dimethylxanthine-Nicotinic acid;
and
a pharmaceutically acceptable carrier.

39. An anti-pulmonary artery hypertension pharmaceutical composition including:
an effective amount of a compound of 7-[2-[4-(2-nitrobenzene)piperazinyl]ethyl]-1,3-dimethylxanthine hydrochloride; and
a pharmaceutically acceptable carrier.

40. An anti-pulmonary artery hypertension pharmaceutical composition including:
an effective amount of a compound of 7-[2-[4-(2-nitrobenzene)piperazinyl]ethyl]-1,3-dimethylxanthine-Citric acid; and
a pharmaceutically acceptable carrier.

41. An anti-pulmonary artery hypertension pharmaceutical composition including:
an effective amount of a compound of 7-[2-[4-(2-nitrobenzene)piperazinyl]ethyl]-1,3-dimethylxanthine-Nicotinic acid;
and
a pharmaceutically acceptable carrier.

42. An anti-pulmonary artery proliferation inhibiting pharmaceutical composition including:

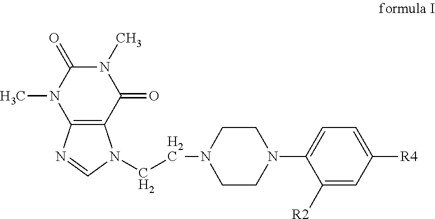

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

43. The pharmaceutical composition of Embodiment 42, wherein the halogen atom is one selected from a group consisting of a fluorine, a chlorine, a bromine and an iodine.

44. The pharmaceutical composition of any one of Embodiments 42-43, wherein the compound of formula I is KMUPs compound.

45. The pharmaceutical composition of any one of Embodiments 42-44, wherein the KMUPs compound is one selected from a group consisting of KMUP-1, KMUP-2, KMUP-3, KMUP-4 and its pharmaceutical acceptable salts.

46. An anti-pulmonary artery proliferation inhibiting pharmaceutical composition, including:
an effective amount of a compound of 7-[2-[4-(2-chlorobenzene)piperazinyl]ethyl]-1,3-dimethylxanthine; and
a pharmaceutically acceptable carrier.

47. An anti-pulmonary artery proliferation inhibiting pharmaceutical composition including:
an effective amount of a compound of 7-[2-[4-(2-methoxybenzene)piperazinyl]ethyl]-1,3-dimethylxanthine; and
a pharmaceutically acceptable carrier.

48. An anti-pulmonary artery proliferation inhibiting pharmaceutical composition including:
an effective amount of a compound of 7-[2-[4-(4-nitrobenzene)piperazinyl]ethyl]-1,3-dimethylxanthine; and
a pharmaceutically acceptable carrier.

49. An anti-pulmonary artery proliferation inhibiting pharmaceutical composition including:
an effective amount of a compound of 7-[2-[4-(2-nitrobenzene)piperazinyl]ethyl]-1,3-dimethylxanthine; and
a pharmaceutically acceptable carrier.

50. An anti-pulmonary artery proliferation inhibiting pharmaceutical composition including:
an effective amount of a compound of 7-[2-[4-(2-chlorobenzene)piperazinyl]ethyl]-1,3-dimethylxanthine hydrochloride; and
a pharmaceutically acceptable carrier.

51. An anti-pulmonary artery proliferation inhibiting pharmaceutical composition including:
an effective amount of a compound of 7-[2-[4-(2-chlorobenzene)piperazinyl]ethyl]-1,3-dimethylxanthine-Citric acid; and
a pharmaceutically acceptable carrier.

52. An anti-pulmonary artery proliferation inhibiting pharmaceutical composition including:
an effective amount of a compound of 7-[2-[4-(2-chlorobenzene)piperazinyl]ethyl]-1,3-dimethylxanthine-Nicotinic acid;
and
a pharmaceutically acceptable carrier.

53. An anti-pulmonary artery proliferation inhibiting pharmaceutical composition including:
an effective amount of a compound of 7-[2-[4-(2-methoxybenzene)piperazinyl]ethyl]-1,3-dimethylxanthine hydrochloride; and
a pharmaceutically acceptable carrier.

54. An anti-pulmonary artery proliferation inhibiting pharmaceutical composition including:
an effective amount of a compound of 7-[2-[4-(2-methoxybenzene)piperazinyl]ethyl]-1,3-dimethylxanthine-Citric acid;
and
a pharmaceutically acceptable carrier.

55. An anti-pulmonary artery proliferation inhibiting pharmaceutical composition including:
an effective amount of a compound of 7-[2-[4-(2-methoxybenzene)piperazinyl]ethyl]-1,3-dimethylxanthine-Nicotinic acid; and
a pharmaceutically acceptable carrier.

56. An anti-pulmonary artery proliferation inhibiting, pharmaceutical composition including:
an effective amount of a compound of 7-[2-[4-(4-nitrobenzene)piperazinyl]ethyl]-1,3-dimethylxanthine hydrochloride; and
a pharmaceutically acceptable carrier.

57. An anti-pulmonary artery proliferation inhibiting pharmaceutical composition including:
an effective amount of a compound of 7-[2-[4-(4-nitrobenzene)piperazinyl]ethyl]-1,3-dimethylxanthine-Citric acid; and
a pharmaceutically acceptable carrier.

58. An anti-pulmonary artery proliferation inhibiting pharmaceutical composition including:
an effective amount of a compound of 7-[2-[4-(4-nitrobenzene)piperazinyl]ethyl]-1,3-dimethylxanthine-Nicotinic acid; and
a pharmaceutically acceptable carrier.

59. An anti-pulmonary artery proliferation inhibiting pharmaceutical composition including:
an effective amount of a compound of 7-[2-[4-(2-nitrobenzene)piperazinyl]ethyl]-1,3-dimethylxanthine hydrochloride;
and
a pharmaceutically acceptable carrier.

60. An anti-pulmonary artery proliferation inhibiting pharmaceutical composition including:
an effective amount of a compound of 7-[2-[4-(2-nitrobenzene)piperazinyl]ethyl]-1,3-dimethylxanthine-Citric acid; and
a pharmaceutically acceptable carrier.

61. An anti-pulmonary artery proliferation inhibiting pharmaceutical composition including:
an effective amount of a compound of 7-[2-[4-(2-nitrobenzene)piperazinyl]ethyl]-1,3-dimethylxanthine-Nicotinic acid; and
a pharmaceutically acceptable carrier.

62. An vascular remodeling inhibiting pharmaceutical composition including:

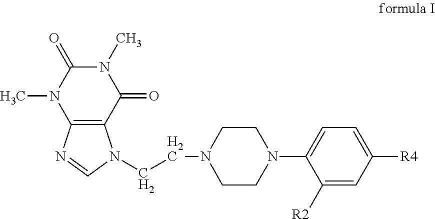

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

63. The pharmaceutical composition of Embodiment 62, wherein the halogen atom is one selected from a group consisting of a fluorine, a chlorine, a bromine and an iodine.

64. The pharmaceutical composition of any one of Embodiments 62-63, wherein the compound of formula I is KMUPs compound.

65. The pharmaceutical composition of any one of Embodiments 62-64, wherein the KMUPs compound is one selected from a group consisting of KMUP-1, KMUP-2, KMUP-3, KMUP-4 and its pharmaceutical acceptable salts.

66. An vascular remodeling inhibiting pharmaceutical composition, including:
an effective amount of a compound of 7-[2-[4-(2-chlorobenzene)piperazinyl]ethyl]-1,3-dimethylxanthine; and
a pharmaceutically acceptable carrier.

67. An vascular remodeling inhibiting pharmaceutical composition including:
an effective amount of a compound of 7-[2-[4-(2-methoxybenzene)piperazinyl]ethyl]-1,3-dimethylxanthine; and
a pharmaceutically acceptable carrier.

68. An vascular remodeling inhibiting pharmaceutical composition including:
an effective amount of a compound of 7-[2-[4-(4-nitrobenzene)piperazinyl]ethyl]-1,3-dimethylxanthine; and
a pharmaceutically acceptable carrier.

69. An vascular remodeling inhibiting pharmaceutical composition including:
an effective amount of a compound of 7-[2-[4-(2-nitrobenzene)piperazinyl]ethyl]-1,3,3-dimethylxanthine; and
a pharmaceutically acceptable carrier.

70. An vascular remodeling inhibiting pharmaceutical composition including:
an effective amount of a compound of 7-[2-[4-(2-chlorobenzene)piperazinyl]ethyl]-1,3-dimethylxanthine hydrochloride; and
a pharmaceutically acceptable carrier.

71. An vascular remodeling inhibiting pharmaceutical composition including:
an effective amount of a compound of 7-[2-[4-(2-chlorobenzene)piperazinyl]ethyl]-1,3-dimethylxanthine-Citric acid; and
a pharmaceutically acceptable carrier.

72. An vascular remodeling inhibiting pharmaceutical composition including:
an effective amount of a compound of 7-[2-[4-(2-chlorobenzene)piperazinyl]ethyl]-1,3-dimethylxanthine-Nicotinic acid; and
a pharmaceutically acceptable carrier.

73. An vascular remodeling inhibiting pharmaceutical composition including:
an effective amount of a compound of 7-[2-[4-(2-methoxybenzene)piperazinyl]ethyl]-1,3-dimethylxanthine; and
a pharmaceutically acceptable carrier.

74. An vascular remodeling inhibiting pharmaceutical composition including:
an effective amount of a compound of 7-[2-[4-(2-methoxybenzene)piperazinyl]ethyl]-1,3-dimethylxanthine-Citric acid; and
a pharmaceutically acceptable carrier.

75. An vascular remodeling inhibiting pharmaceutical composition including:
an effective amount of a compound of 7-[2-[4-(2-methoxybenzene)piperazinyl]ethyl]-1,3-dimethylxanthine-Nicotinic acid; and
a pharmaceutically acceptable carrier.

76. An vascular remodeling inhibiting pharmaceutical composition including:
an effective amount of a compound of 7-[2-[4-(4-nitrobenzene)piperazinyl]ethyl]-1,3-dimethylxanthine hydrochloride; and
a pharmaceutically acceptable carrier.

77. An vascular remodeling inhibiting pharmaceutical composition including:
an effective amount of a compound of 7-[2-[4-(4-nitrobenzene)piperazinyl]ethyl]-1,3-dimethylxanthine-Citric acid;
and
a pharmaceutically acceptable carrier.

78. An vascular remodeling inhibiting pharmaceutical composition including:
an effective amount of a compound of 7-[2-[4-(4-nitrobenzene)piperazinyl]ethyl]-1,3-dimethylxanthine-Nicotinic acid; and
a pharmaceutically acceptable carrier.

79. An vascular remodeling inhibiting pharmaceutical composition including:
an effective amount of a compound of 7-[2-[4-(2-nitrobenzene)piperazinyl]ethyl]-1,3-dimethylxanthine hydrochloride; and
a pharmaceutically acceptable carrier.

80. An vascular remodeling inhibiting pharmaceutical composition including:
an effective amount of a compound of 7-[2-[4-(2-nitrobenzene)piperazinyl]ethyl]-1,3-dimethylxanthine-Citric acid;
and
a pharmaceutically acceptable carrier.

81. An vascular remodeling inhibiting pharmaceutical composition including:
an effective amount of a compound of 7-[2-[4-(2-nitrobenzene)piperazinyl]ethyl]-1,3-dimethylxanthine-Nicotinic acid; and
a pharmaceutically acceptable carrier.

What is claimed is:

1. An obstructive pulmonary disease inhibiting pharmaceutical composition comprising: 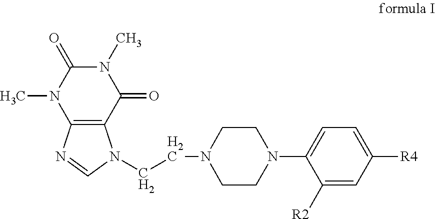
an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

2. A pharmaceutical composition as claimed in claim 1, wherein the halogen atom is one selected from a group consisting of a fluorine, a chlorine, a bromine and an iodine.

3. A pharmaceutical composition as claimed in claim 1, wherein the compound of formula I is KMUPs compound.

4. A pharmaceutical composition as claimed in claim 3, wherein the KMUPs compound is one selected from a group consisting of KMUP-1, KMUP-2, KMUP-3, KMUP-4 and its pharmaceutical acceptable salts.

5. An anti-pulmonary artery hypertension pharmaceutical composition comprising: 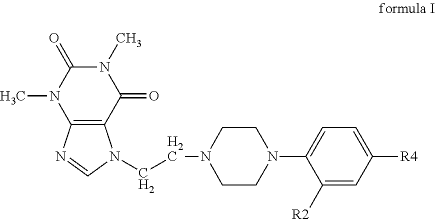
an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

6. A pharmaceutical composition as claimed in claim 5, wherein the halogen atom is one selected from a group consisting of a fluorine, a chlorine, a bromine and an iodine.

7. A pharmaceutical composition as claimed in claim 5, wherein the compound of formula I is KMUPs compound.

8. A pharmaceutical composition as claimed in claim 7, wherein the KMUPs compound is one selected from a group consisting of KMUP-1, KMUP-2, KMUP-3, KMUP-4 and its pharmaceutical acceptable salts.

9. A pharmaceutical composition as claimed in claim 5, wherein the compound is for treating acute or chronic pulmonary artery hypertension.

10. An vascular remodeling inhibiting pharmaceutical composition comprising: 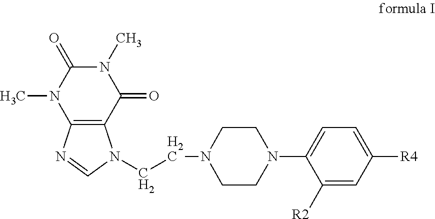
an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

11. A pharmaceutical composition as claimed in claim 10, wherein the halogen atom is one selected from a group consisting of a fluorine, a chlorine, a bromine and an iodine.

12. A pharmaceutical composition as claimed in claim 10, wherein the compound of formula I is KMUPs compound.

13. A pharmaceutical composition as claimed in claim 12, wherein the KMUPs compound is one selected from a group consisting of KMUP-1, KMUP-2,
KMUP-3, KMUP-4 and its pharmaceutical acceptable salts.

14. An obstructive pulmonary disease inhibiting pharmaceutical composition comprising: 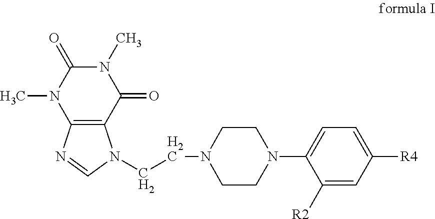
an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

15. A pharmaceutical composition as claimed in claim 14, wherein the halogen atom is one selected from a group consisting of a fluorine, a chlorine, a bromine and an iodine.

16. A pharmaceutical composition as claimed in claim 14, wherein the compound of formula I is KMUPs compound.

17. A pharmaceutical composition as claimed in claim 16, wherein the KMUPs compound is one selected from a group consisting of KMUP-1, KMUP-2, KMUP-3, KMUP-4 and its pharmaceutical acceptable salts.
